(19) United States
(12) Patent Application Publication
(10) Pub. No.: US 2008/0292715 A1
(43) Pub. Date: Nov. 27, 2008

(54) Unique Manuka Factor (Umf) Fortified Honey

(75) Inventors: Melanie Jane Snow, Auckland (NZ); Merilyn Manly-Harris, Hamilton (NZ); Judie Marie Farr, Auckland (NZ)

Correspondence Address: KNOBBE MARTENS OLSON & BEAR LLP, 2040 MAIN STREET, FOURTEENTH FLOOR, IRVINE, CA, 92614, US

(73) Assignee: WAIKATO THE UNIVERSITY OF, HAMILTON (NZ)

(21) Appl. No.: 11/628,882

(22) PCT Filed: Jun. 8, 2005
(86) PCT No.: PCT/NZ05/00118

§ 371 Date: Mar. 5, 2008

(30) Foreign Application Priority Data
Jun. 8, 2004 (NZ) 533368

(51) Int. Cl. A61K 35/64; A23L 1/08; A01P 1/00

(52) U.S. Cl. 424/539; 426/658

(57) ABSTRACT

The invention relates to UMF amended food stuffs and medicaments. In particular, although not exclusively, the invention relates to UMF fortified honey, methods for the preparation of UMF fortified honey, and methods for the preparation of UMF containing fractions of honey.

### FIELD OF INVENTION

The invention relates to UMF amended food stuffs and medicaments. In particular, although not exclusively, the invention relates to UMF fortified honey, methods for the preparation of UMF fortified honey, and methods for the preparation of UMF containing fractions of honey.

### BACKGROUND OF THE INVENTION

The manuka tree is native to New Zealand and yields a honey that has a very strong flavour. Besides being used as food for millennia, honey has also been used for medicinal purposes, principally as an antibacterial agent. The major cause of the antibacterial activity is due to hydrogen peroxide that is produced in honey by the enzyme glucose oxidase. Manuka honey has been found to possess an amount of activity that is in addition to this antibacterial activity. This additional activity is known as the non-peroxide activity and is commercially known as unique manuka factor (UMF).

There have been many studies identifying properties of the non-peroxide activity and attempts to identify the fraction responsible for this additional activity have also been made but have so far been unsuccessful.

Honey has been used for millennia with the first written documentation dealing with honey dated about 4000 years ago. Honey is the only sweetening material that requires no manipulation or processing to render it ready to eat (White, 1992).

The manuka tree, Leptospermum scoparium, is native to New Zealand. It favours wetter and low-fertility leached soils, and lives to about 60 years. The tree can grow to 6-8 m in height and 7-10 cm in diameter. It has flowers which are 10-12 mm across and they are generally white (Ward, 2000). Honey yielded from the manuka tree has a strong flavour with a herby, woody characteristic and is dark in colouring.

There have been many studies carried out to identify the compounds present in honey. Reasons include to create a ‘fingerprint’ database for honeys so that geographical and floral origin can be determined, and also to detect honey adulteration.

Most of the compounds are included within the classes of carbohydrates, enzymes, aromatic acids, hydrocarbons, straight-chained mono- and di-basic acids and water.

While honey is composed mainly of the sugars glucose, fructose, sucrose and maltose (White, 1978), many other oligosaccharides have also been identified.

Significant oligosaccharides identified in manuka honey include maltulose, kojibiose, turanose, nigerose, maltose, trehalose, palatinose, sucrose, erlose and panose, melezitose and maltotriose (Weston & Brocklebank, 1999; Wu, 2000). Wu (2000) found turanose to be the principal oligosaccharide in manuka honey where as Weston & Brocklebank (1999) found maltose to be the principal oligosaccharide.

Other substances added to the honey by the bee are amino acids, the most abundant being proline, and minor amounts of catalase (White, 1992). The enzyme catalase degrades hydrogen peroxide into water and oxygen.

Manuka honey contains high concentrations of aromatic acids with the dominant being 2-hydroxy-3-phenylpropionic acid (Tan et. al., 1988; Wilkins et. al., 1993) and the aromatic acids are in a much greater concentration in manuka than New Zealand clover honey (1000 times greater) (Tan et. al., 1988). Aromatic acids also identified have been 2-methoxybenzoic acid, 2′-methoxyacetophenone, 2-decenedioic acid, 4-hydroxy-3-5-dimethoxybenzoic acid, 2-hydroxy-3-(4′-methoxyphenyl)propionic acid, syringic acid, 3,4,5 trimethoxybenzoic acid and acetophenone (Tan et. al., 1988; Wilkins et. al., 1993).

Irrespective of the season and geographical origin, samples of unifloral manuka honey can be characterised by the combined concentrations of the propionic acids being greater than 700 mg/kg honey, the benzoic acids combined being greater than 35 mg/kg honey, and the acetophenones being greater than 20 mg/kg honey combined (Wilkins et. al., 1993).

Tan et. al. (1989) also identified 2-hydroxy-3-phenylpropionic acid as a major characteristic compound and illustrated the use of the higher levels of this compound along with 2-methoxybenzoic acid and 2′-methoxyacetophenone as markers to determine manuka floral origin.

Wilkins et. al. (1993) states that decanedioic acid and trans-2-decenedioic acid are often found in higher concentrations in manuka honey than other honey. Diacids including octandedioic, nonanedioic, decanedioic, and trans-2-decenedioic have also been reported in honey (Tan et. al., 1988; Wilkins et. al., 1993).

Other compounds identified in other honeys by White et. al. (1962) included lactone, diastase, free acidity and ash. The literature does not contain any information on these in manuka honey, nor is there any published information on the moisture content of manuka honey.

Honey has been used as a medicine by many cultures since ancient times (Ransom, 1937, Adcock, 1962). More recently the interest in honey as a newsworthy agent has increased due to the recognition of a definite antibacterial effect. This antibacterial effect of honey varies substantially depending on honey type (Dustmann, 1979).

It has also been observed that manuka (Leptospermum scoparium: Myrtacaea) honey has a higher level of antibacterial activity that cannot be explained solely by the honey osmolarity, pH and glucose oxidase activity (Russell, 1983). This contributor to activity is now referred to as the “non-peroxide activity” or “UMF”.

Dustman (1979) had noted the existence of antibacterial activity that was not due to glucose oxidase activity or the high osmolarity. However, he was of the opinion that the latter activity was only a minor portion of the total activity.

Molan & Russell (1988) gave evidence for the existence of antibacterial activity not due to hydrogen peroxide in manuka honey and also found that manuka honeys with a high overall activity had a high amount of the non-peroxide activity.

Studies into the non-peroxide activity of honey have found that it has some interesting and apparently contradictory properties. Verge (1951) obtained active fractions in water, alcohol, ether and acetone. Schuler and Vogel (1956) extracted the activity with ether. Lavie (1960) found activity extraction possible with acetone but not ether. Gonnet and Lavie (1960) found activity in cold ether extract was volatile at 95° C. Mladenov (1974) reported that honey contains volatile, heavy-volatile and non-volatile antibacterial substances.

The manuka honey activity has been found to be heat and light stable (Molan & Russell, 1988, Tan et. al., 1988, Russell et. al., 1990), and preliminary studies of Tan et. al. (1988) showed that the additional activity was soluble in organic solvents, e.g. ethanol and ether.

Aromatic acid and phenolic compounds are the only other substances with antibacterial activity apart from the hydrogen peroxide and the enzyme lysozyme that have been isolated from honey (Weston et. al., 2000).

Caffeic and ferulic acid, both phenolic acids, have been identified as possessing antimicrobial activity (Cizmarik and Matel, 1970, Cizmarik and Matel, 1973) and have been isolated from honey (Wahdan, 1998, Weston, 2000). However, they are found only in low concentrations and contribute little to the antibacterial activity of honey when compared with the contribution from hydrogen peroxide (Weston, 2000).

Marhuenda Requenda et. al. (1987) reported antibacterial activity of other phenolic acids including caffeic, vanillic, p-coumaric, p-hydroxybenzoic and syringic acids.

The flavonoids isolated from honey include pinocembrine, pinobanksin, chrysin and flavonone (Wahdan, 1998, Ferreres et. al., 1994). These have well documented antimicrobial activity (Rivera-Vargas et. al., 1993, Itoh et. al., 1994) but also do not occur at high enough concentrations in honey to have a significant antibacterial effect (Weston, 2000).

Russell (1983) identified 2 aromatic compounds as major antibacterial components from honey. They were 4-hydroxy-3-5-dimethoxybenzoate and methyl-3,4,5-trimethoxybenzoate.

Propolis, a resinous material collected by bees from the gum exudates of trees and used as an antibacterial agent in the hive, includes substituted benzoic and cinnamic acids and flavonoids (Marcucci, 1995). Wahdan (1998) states that flavonoids are the major substances in antibacterial propolis.

The components found responsible for the antibacterial activity have been identified as galangin, pinocembrin, caffeic acid and ferulic acid. (Lavie, 1960, Ghisalbert, 1979, Russell et. al., 1990). Weston (2000) has since demonstrated that the individual components lacked antimicrobial activity, but bioactivity was only observed for whole propolis.

Mellitin and phospholipase are components of bee venom thought to be responsible for its weak antibacterial activity. Both of these are proteins and gel filtration chromatography has shown that the antibacterial substances in manuka honey have a molecular weight less than 1000 amu (Russell et. al., 1990).

Other products that could contribute to the non-peroxide activity of manuka honey could be antibiotic peptides characterised from the body fluid of bees. Those identified have been abaecin, apidaecin, hymenoptaecin, royalism and lysozymes. The peptides possess strong antibiotic activity and if they were present in honey they could contribute significantly to the non-peroxide activity (Weston et. al., 2000).

Despite many years of research the non-peroxide component of the antibacterial activity of manuka honey (UMF) has not been isolated or identified. Indeed some researchers do not consider an isolatable fraction of component of manuka honey to exist.

Russell et. al. (1990) considered it likely that the difference in opinion on the significance of the additional antibacterial activity (i.e. that not due to hydrogen peroxide or high osmolarity) results from the difference that exists in the amount of this activity. Molan and Russell (1988) found that for a range of New Zealand honey samples, the additional activity varied from nil in some samples to almost the whole of the activity in other samples. They also noted a close correlation existed between the level of additional antibacterial activity and the overall antibacterial activity of individual honey samples.

Bogdanov (1997) attempted to determine in which fraction the non-peroxide activity occurred by separating the volatile, non-polar and non-volatile, acidic, and basic fractions. He did this by testing the activity of a sample, removing a fraction, and then testing the activity again. If the activity did not remain in the sample then he concluded that the antibacterial constituent was present in the removed fraction. Extraction of volatile substances was done by heating at 60° C. in a Rotovapor for 2 hours. Extraction of non-polar, non-volatile substances was done by C-18 columns. Extraction of bases was done on a cation exchange column in the H form and extraction of acids was done on an anion exchange column in the OH form. For acid removal the honey was set to pH 11 so acids were in the dissociated anion form.

Bogdanov stated that the shift in initial honey pH to pH 11 had no effect on the antibacterial activity as back titration to the original pH resulted in the restoration of the initial antibacterial activity. For manuka honey, Bogdanov stated that 100% of the activity was in the acidic fraction when tested against Staphylococcus aureus and when tested against Micrococcus luteus, 75% was in the acidic fraction, 10% in the basic fraction, 5% in the non-polar fraction, and 10% in the volatile fraction. It was hence concluded that the non-peroxide antibacterial activity in honey was found in the acid fraction and correlated significantly with the acid content, but not with pH.

Wahdan (1998) found by comparing the osmotic effect of sugar solutions to undiluted honey, that the high sugar concentration is an important factor in the antimicrobial activity but when it is diluted it becomes apparent there are other contributors also present. It was also stated that in this study pH could not have been a factor because the honey was diluted by nutrient broth (pH 7.2) so there must have been other substances present.

Weston & Brocklebank (1999) attempted to separate the monosaccharides from the antibacterial material by testing several methods that included chromatography on poly(capryl)amide, Sephadex G-10, Biogel P-2, and XAD-2 resin in both acidic and neutral conditions. Isolation with 2-butanol was also tested. The chromatography on polyamide and Sephadex G-10 indicated that the active material was located in late fractions and analysis by HPLC suggested flavonoids were present.

The major product in the phenol extract was identified as methyl syringate by thin-layer chromatography and a minor product was phenyllacetic acid. HPLC analysis showed that methyl syringate constituted more than 45% of the total phenolic extract. This paper also tested and concluded that at the level of methyl syringate and phenyllacetic acid in manuka honey they themselves did not account for all of the non-peroxide activity. Furthermore the level of methyl syringate was the same in both active and non-active manuka honey and therefore could not be responsible for the observed differences.

On XAD-2 all of the activity was eluted with carbohydrates and no significant activity with the phenolics. The same result was obtained with Biogel-P2. Since the monosaccharides have no antibacterial properties it was suggested that the antibacterial component is being carried by the sugars.

Weston et. al. (1999) demonstrated that the phenolic fraction as a whole had the same antibacterial effect between active and non active honeys so concluded that while the phenolic components of manuka honey individually and collectively were antibacterially active, they were not responsible for the ‘observed’ activity.

Weston & Brocklebank (1999) hypothesised that active manuka honey may have had a unique oligosaccharide similar to the tetrasaccharide sialyl Lewis X which is the antigenic determinant which mediates the adhesion of the bacterium that causes stomach ulcers, to the lining of the stomach. They tested the oligosaccharide composition of active and non-active manuka honeys and found no differences between active and inactive manuka honey.

Perry et. al. (1997) discovered three chemotypes of manuka existed in New Zealand and that they can be distinguished by the composition of the essential oil from the leaves. One contains a high portion of pinenes, another a high portion of sesquiterpenes and the third, which grows in the Eastland region, contains a high proportion of a cyclictriketone, leptospermone. This oil had the greatest antimicrobial activity of the three.

Weston et. al. (2000) attempted to detect leptospermone in a sample of very high non-peroxide activity manuka honey by three different methods. One was using XAD-2 resin to adsorb the phenolics, another was using extraction with 2-butanol, and the third was liquid-liquid extraction. Analysis of all three extracts failed to detect any triketones. It was concluded that because leptospermone is insoluble in water it is unlikely to be present in nectar and consequently not in honey.

Weston et. al. (2000) compared the phenolic components in the antibacterial manuka honey and inactive manuka honey and showed that there was no difference qualitatively or quantitatively. The levels of cinnamic acid and flavonoids were also very similar and comparable with many European honeys. By testing 19 manuka honeys they showed that the phenolic profile was identical across all samples hence geography does not influence the phenolic profile.

Weston (2000) stated that:
(i) hydrogen peroxide is the only antibacterial substance of any consequence in honey and that other substances such as propolis-derived phenolics are insignificant in comparison to hydrogen peroxide;
(ii) the level of hydrogen peroxide in a honey is essentially determined by the amount of plant derived catalase in the honey; and
(iii) based on the findings of White et al. (1963) and Dustmann (1971), hydrogen peroxide is generated by glucose oxidase in samples of honey or fractions thereof, when they are diluted and prepared for antibacterial assays the amount of catalase added to these samples in present methods (Allen, Molan and Reid, 1991, Molan and Russell, 1988) is insufficient to destroy all of the hydrogen peroxide produced in this way.

It was also stated that the volatiles analysed by GC-MS are, in general, components of nectar and contribute to the aroma of flowers. Although there are a wide variety of them, their quantities in honey are small and the components that are unique to a particular flower source and honey do not appear to have any antibacterial properties at their level in honey. Also the phenolic components of nectar used to identify the honey as unifloral, do have antioxidant properties but have not been identified as having appropriate levels of antibacterial activity.

Weston (2000) therefore concludes that work to date discounts the possibility of the existence of UMF and suggests it is more likely that it is due to an unusually high level of hydrogen peroxide that is incompletely destroyed by the addition of the catalase. The “unique factor” could in fact be the presence or absence of catalase in large amounts in manuka honey that differentiates active and non-active manuka honey.

Honey with a high UMF value, and products derived from such honey, are sought after by consumers. This is irrespective of whether the beneficial properties of honey with a high UMF value are attributable to an isolatable UMF containing fraction.

If a fraction of manuka honey associated with the UMF activity could be isolated UMF fortified honey and other amended foodstuffs and medicaments could be prepared. The known favourable properties and beneficial effects of manuka honey could be augmented.

It is an object of this invention to provide a method for the preparation of a UMF containing fraction of manuka honey, or to at least provide the public with a useful choice.

### STATEMENT OF INVENTION

In a first aspect the invention provides UMF fortified honey.

Preferably the honey is manuka honey.

Preferably the honey has a UMF value greater than that of any unfortified manuka honey. More preferably the fortified honey has a UMF value greater than 25, more preferably greater than 35.

In a second aspect the invention provides a method of preparing a UMF fortified honey including the step of mixing a honey with a UMF containing fraction.

In a third aspect the invention provides a method of preparing a UMF containing fraction by separating the fraction from substantially all of the total monosaccharide sugars in the sample from which the fraction is obtained.

Preferably the method includes the steps of:
applying an amount of honey to a matrix;
eluting the sample from the matrix with a solvent; and
collecting the UMF containing fraction.

Preferably the collecting the UMF containing fraction commences following elution of substantially all the monosaccharide sugars present in the sample. More preferably the collected UMF containing fraction is substantially free of the total amount of monosaccharide sugars present in the sample.

Preferably the honey is manuka honey.

Preferably the amount of manuka honey has a UMF value greater than 25, more preferably greater than 35.

Preferably the matrix is a size exclusion matrix or a reverse phase matrix.

Preferably the solvent is water.

Preferably the matrix is in the format of a chromatography column.

In a first embodiment of the third aspect of the invention the matrix is a size exclusion and ion exchange matrix. Preferably the counter ion is Na⁺. More preferably the matrix has a size exclusion limit of 10⁴. More preferably the matrix is styrene divinylbenzene copolymer.

In a second embodiment of the third aspect of the invention the matrix is C18.

Preferably the matrix has a 15 μm particle size and a 100 Å pore size.

While the use of whole honey is preferred, components or portions previously separated from honey, including sieved honey, may also be used in the method.

In a fourth aspect the invention provides a UMF containing fraction of manuka honey.

Preferably the fraction is substantially free of monosaccharide sugars.

Preferably the antibacterial activity of the UMF containing fraction is labile at alkaline pH. More preferably the alkaline pH is greater than 9.

Preferably the UMF containing fraction is prepared by the method according to the third aspect of the invention.

Preferably the UMF containing fraction has the chromatographic characteristics described in Examples 2, 3 and 4.

Preferably the UMF containing fraction has a retention time of 19.4 to 25 minutes when a sample (20 μL) of honey containing the UMF containing fraction is applied to Shodex™ Sugar KS-801 and KS-802 analytical columns in series and in the sodium form, operated at a temperature of 50° C. and eluted with Milli-Q water at a rate of 1 mL/min. More preferably the UMF containing fraction has a retention time of 19.4 to 21.7 minutes.

In a fifth aspect the invention provides a medicament amended with the UMF containing fraction according to the fourth aspect of the invention.

Preferably the medicament is a wound dressing, such as that described in New Zealand patent application no. 501687.

In a sixth aspect the invention provides a food stuff amended with the UMF containing fraction according to the fourth aspect of the invention.

Preferably the food stuff is honey.

“UMF fortified honey” means a honey to which an isolated UMF containing fraction has been added.

“Manuka honey” means a floral honey derived predominantly from the flowers of manuka (Leptospermum scoparium).

“UMF value” means the measurement of antibacterial activity determined for a whole or sieved honey or fraction thereof determined relative to phenol equivalents in an agar plate diffusion assay.

“UMF containing fraction” means a fraction of whole or sieved manuka honey containing a non-peroxide antibacterial activity.

“Substantially free of monosaccharide sugars” in respect of a fraction means the amount of monosaccharide sugars by weight in the fraction is a small or negligible portion of the total monosaccharide sugars in the sample from which the fraction is obtained, e.g. less than 5% (w/w) of the total monosaccharide sugars, more typically less than 1% (w/w).

It is recognized the adoption of the invention may allow the unequivocal detection of a UMF containing fraction in whole honey not previously known to exhibit non-peroxide activity. It is further recognized that this non-peroxide activity may be exhibited by honeys other than manuka honey and not yet tested for the presence of a UMF containing fraction.

### BRIEF DESCRIPTION OF THE DRAWINGS

The invention will now be described in detail with reference to the accompanying drawings in which:

FIG. 1 HPLC refractive index analysis of manuka honey using ligand exchange and size exclusion chromatography.

FIG. 2 Enlarged base line region of a HPLC refractive index analysis of manuka honey by ligand exchange and size exclusion chromatography.

FIG. 3 HPLC of sieved honey on KS-2002 column. Refractive index monitoring of the eluant from a 200 μl injection of 300 mg/mL sieved honey.

FIG. 4. HPLC of the re-injected active fraction from the KS-2002 column. Refractive index monitoring of the eluant from a 200 μl injection of fraction 4 (at a concentration that would be equivalent to that which would occur in 200 mg/ml sieved honey.)

FIG. 5. HPLC of the re-injected active fraction from the KS-2002 column. Enlargement of the refractive index monitoring of the eluant from a 200 μL injection of fraction 4 (at a concentration equivalent to that which would occur in 200 mg/mL of sieved honey).

FIG. 6. HPLC of the 20.5 to 25 min fraction from fraction 4 on the KS-2002 column. Refractive index monitoring of the eluant from a 200 μL injection (at a concentration equivalent to that which would occur in 100 mg/mL of sieved honey)

FIG. 7. HPLC spectrum of the 20.5 to 25 min fraction from the re-injection of fraction 4 on the KS-2002 column. Enlargement of the refractive index monitoring of the eluant from a 200 μL injection of this fraction (at a concentration equivalent to that which would occur in 100 mg/mL of sieved honey)

FIG. 8. HPLC on the C18 column. Refractive index monitoring of the eluant from a 2 ml injection of 0.5 g/mL sieved honey.

FIG. 9. HPLC on the C18 column. Enlargement of the refractive index monitoring of the eluant from a 2 mL injection of 0.5 g/mL sieved honey.

FIG. 10. Demonstration of the fractions collected in Milli-Q and MeCN from the C18 column.

FIG. 11. Effect of storage on the non-peroxide activity of the active fraction.

FIG. 12. Investigation of the proportionality of fortifying sieved honey.

FIG. 13. Data from the fortifying experiment grouped by plate. (1̂2 is the zone of clearing, in mm², and ratio is the ratio of fraction C to sieved honey, in g/g)

### DETAILED DESCRIPTION

The invention is the determination that a small fraction of the honey, which typically makes up less than one percent of the dry weight, contains virtually all the non-peroxide activity and that this can be isolated from the bulk of the honey source as a UMF containing fraction.

The UMF containing fraction may be separated from the bulk of the honey by a number of means known to a skilled addressee. However, while previous attempts have been made using HPLC to separate and isolate the agents which confer the non-peroxide bioactivity on honey, the choice of columns and solvent used in the prior art have prevented this goal from being realised.

The prior art attempts have likely either unknowingly destroyed the bioactivity of the honey by using the wrong experimental conditions or used columns which did not allow adequate separation and thus the identification of separate fractions.

Further, because the UMF fraction is a small portion of the honey, it is also likely that if separation had previously occurred the elution peak could have been unwittingly ignored or disguised by other compounds.

In the preferred embodiments of the invention separation columns designed to separate the principal monosaccharides present in honey are used. These principal monosaccharides are glucose and fructose. Examples of columns include Rezex™, Nucleosil™ and Shodex™. It should be appreciated that these are given by way of example only.

During the elution of honey through the columns, small fractions are preferably collected and analysed using one and/or both UV absorption and refractive index detection to monitor the elution of fractions of interest.

Shodex™ (mixed mode ligand exchange and size exclusion) chromatographic columns have been used to separate glucose and fructose from other components of the honey. It has been discovered that a fraction detected as a small peak by refractive index monitoring is eluted after the fructose peak.

This fraction has a UV absorption whereas fructose does not. This fraction has been tested and found to contain virtually all the non-peroxide antibacterial activity of the honey. This fraction is referred to as the UMF containing fraction. When using catalyse to destroy any peroxide-based antibacterial activity of honey the activity of the UMF fraction is maintained.

Liquid-liquid extraction of honey using ether could also be used to simplify the isolation of the UMF containing fraction. In these experiments, ether can remove many of the non-sugar, organic compounds from the honey, while maintaining the non-peroxide activity in the remaining sample.

In order to further purify the UMF fraction, it may be passed through the columns multiple times to eliminate more and more impurities or subjected to chromatography on columns with other types of packing resins, for example reversed-phase columns, which might separate the components of the fraction.

The UMF fraction has never previously before been separated, or even recognised to exist as a discrete faction despite multiple attempts by a number of different research groups. Reversed-phase, Bio-Gel P-2, XAD-40 and anion-exchange columns have all been previously used in the prior art in attempts to isolate and identify compounds with non-peroxide bioactivity.

Under these conditions either the fraction was not separated or was not recognised because of its small size, perhaps being disguised by the large monosaccharide peaks in previous HPLC honey studies or the chromatography conditions destroyed the bioactivity of the honey.

Preliminary investigations by the inventors have found that separation of the UMF fraction cannot occur on columns that require alkaline pH, such as anion-exchange columns. Alkaline conditions were found to destroy the activity of honey. Thus columns must be used that run at or near neutral pH.

The UMF containing fraction was found to lose most of its bioactivity after 30 minutes at pH 9. The fraction was destroyed after 5 minutes at pH 10. HPLC separations routinely take longer than this. At pH 11 the bioactivity of honey is immediately destroyed.

Manuka honey samples showing non-peroxide activity (UMF) were extracted with ether using standard techniques to remove fatty acids that could interfere with chromatography. Both the aqueous and ether phases were tested by using antibacterial assays to determine in which phase the UMF activity was isolated. The UMF activity was found to remain in the aqueous phase, with no significant bioactivity found in the ether phase.

To examine the level of non-peroxide activity in the honey samples catalase was used to destroy any antibacterial activity conferred to honey by hydrogen peroxide. Catalase solution was made by dissolving catalase (0.02 g) in distilled water (10 mL). Honey was dissolved in distilled water at a concentration of 1 gram honey/mL water, in 1 mL aliquots. Then either 1 mL of distilled water (for total activity) or 1 mL of catalase solution (for non-peroxide activity alone) was added to each sample vial.

### EXAMPLE 1

### Well Diffusion Assay of Antibacterial Activity

Nutrient agar was prepared by dissolving agar (23 g) in distilled water (1 L) and pouring 150 mL amounts into flasks before autoclaving. When required, flasks were steamed in a water bath (30 min, 100° C.), and then the agar temperature was reduced in another water bath to a temperature tolerable for the bacterial culture (30 min, 50° C.).

The S. aureus culture was produced by aseptically inoculating tryptic soy broth (30 g/L) with a bead culture and then incubating (18 h, 37° C.). The S. aureus culture was adjusted to an optical density of 0.5AU with tryptic soy broth, using a Thermo Spectronic Helios γ spectrophotometer (540 nm). Tryptic soy broth was used as the blank.

Large squared assay plates (Corning® 431111 sterile bioassay dish, 245 mm×245 mm×18 mm) were prepared on a level surface by pouring 150 mL of nutrient agar seeded with S. aureus culture (100 μL, 0.5 OD as prepared above). Once solidified, the plates were stored upside-down at 4° C. overnight.

Whole honey samples were prepared by weighing whole honey (1.00 g) and dissolving in distilled water (1 mL). For the reproducibility assays, this process was aided by incubation (37° C., 30 min) to soften the honey, while keeping the time available for H₂O₂ production constant. For other experiments, where H₂O₂ activity was not being determined, the samples were stirred at room temperature until dissolved.

The resulting 50% (w/v) solution was further diluted by combining equal volumes (1:1) of sample with either distilled water or catalase solution (20 mg/10 mL distilled water) depending on whether total non-peroxide activity is required.

Honey fractions from HPLC and liquid/liquid extractions were dissolved (1:1) in distilled water to give the same concentration that was present prior to separation of the honey. The resulting 50% solution was further diluted (1:1) with catalase solution as only non-peroxide activity was of interest.

Phenol standards of 2, 3, 4, 5, 6, and 7% were prepared from a 10% stock solution of phenol (10 g phenol/100 mL distilled water). These were stored in the dark at 4° C. for up to one month before being replaced.

Wells were punched in a regular 8×8 grid using an 8 mm cork borer and inoculating needle to remove agar. The template used for placing the samples on the plate was a Quasi-Latin square with 16 numbered wells repeated 4 times over the plate, once in each pair of rows and columns. This allowed samples to be placed randomly on the plate to remove bias from edge effects.

Honey samples at 25% concentration and phenol standards were placed in each well (11 μL), at allocated positions.

### EXAMPLE 2

High performance liquid chromatography (HPLC) was conducted on a Waters HPLC system using a 515 HPLC pump, a 2410 refractive index detector, a 996 photodiode array detector and Millennium operating software.

In initial studies Shodex™ Sugar KS800 series columns were found to provide the best separation out of all the columns used. Here, Shodex™ Sugar KS801 and KS 802 were used in series to fractionate the honey samples by combined size exclusion and ligand exchange chromatography.

The KS801 was in the sodium form with an exclusion limit of 10³. KS802 was also in the sodium form and had an exclusion limit of 10⁴. Both were packed with styrene divinylbenzene. The operating temperature used was initially 80° C. with a flow rate of 1 mL/min as suggested by the manufacturer. The eluant was Milli-Q water.

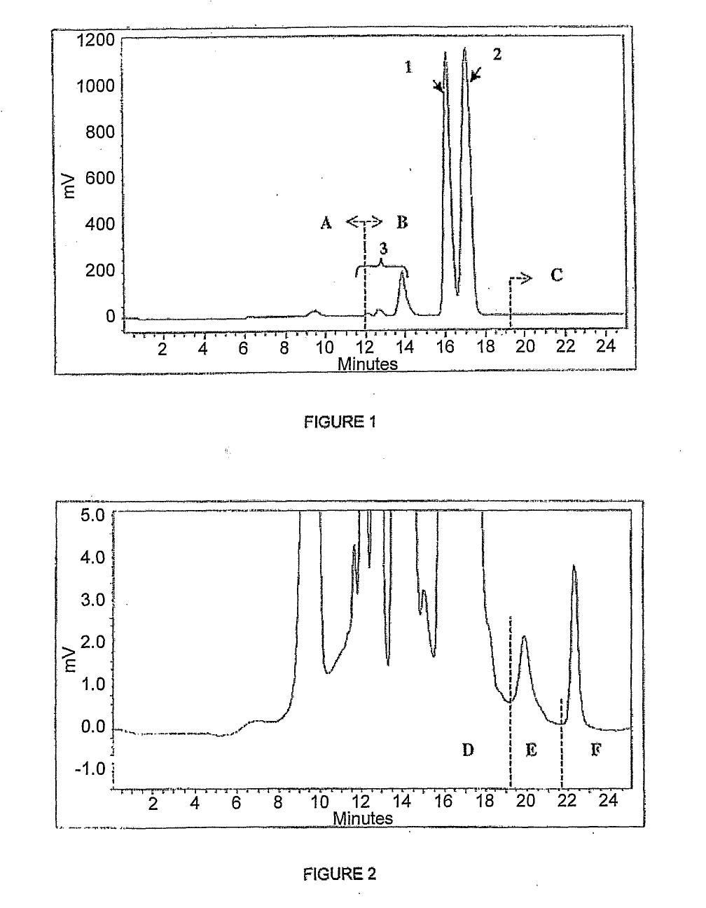

Honey samples of 20 mg/20 μL injection were loaded onto the KS800 series HPLC columns. FIGS. 1 and 2 show the plots obtained with refractive index detection. Fractions were collected for antibacterial assay from 20 injections.

In FIG. 1, the fraction A was collected from 0 to 12 minutes, fraction B was collected between 12 and 19.4 minutes, and fraction C was collected from 19.4 to 25 minutes. The plot shows the glucose (1) peak followed by the fructose (2) peak. An oligosaccharide (3) peak is also shown.

The antibacterial activity of the separate fractions was tested using the well diffusion technique using Staphylococcus aureus as the test culture.

Fractions collected from the HPLC for testing were evaporated under reduced pressure on a Büchi RE111 Rotovapor coupled with a BOchi 461 water bath at 40° C. The samples were then re-dissolved in a solution containing 200 μL of distilled water and 200 μL of catalase solution to ensure only non-peroxide activity was present.

Honey samples were tested in a concentration of 25% for antibacterial activity. The antibacterial assays were conducted using three replicates of the phenol standards ranging from 2% to 6% and three to five replicates of the samples being tested were introduced into recorded random wells in the agar plates.

The plates were incubated at 37° C. overnight allowing the bacteria to grow where possible. After incubation, digital callipers were used to measure the diameter of the area of inhibition around the wells.

The non-peroxide antibacterial activity of the honey was completely contained within fraction C (FIG. 1).

When focussing in on the baseline region of the HPLC plot, two peaks were visible in the active region of fraction C (FIG. 2). To determine which of these peaks was responsible for the activity, another scheme of fraction collection times was devised: fraction D 0 to 19.4 minutes, fraction E 19.4 to 21.7 minutes, and fraction F 21.7 to 25 minutes.

In these experiments, all the antibacterial activity was isolated in fraction E.

This test was repeated a further two times and the same results obtained.

### EXAMPLE 3

### Semi-Preparative Fractionation of Honey (Combined Size Exclusion and Ion Exchange)

Previous studies (Example 2 and Snow (2001)) determined that a UMF containing fraction could be resolved from a substantial portion of the monosaccharide sugars using Shodex™ SUGAR KS-801 and KS-802 analytical columns in series.

A preparative version of this column (a KS-2002 column) was used as it was desirable to increase the amount of sample that could be passed through the column. The elution profile of sieved honey is characterised by early phenolic material, followed by di- and oligosaccharides and finally monosaccharides.

The Shodex™ SUGAR KS2002 (20 mm×300 mm, 20 μm particle size, 60 Å pore size) preparative scale column combines size exclusion and ligand exchange. The column was in the sodium (Na⁺) form and had a 1×10⁴ exclusion limit. Chromatography was performed at room temperature using Milli-Q water as the eluent, running at a flow rate of 3 mL/min.

High loadings of 300 mg/mL honey were injected into a 200 μL loop. The spectrum was monitored using both 996 PDA and 2410 RI detection. Any collected fractions were freeze-dried in large evaporating dishes.

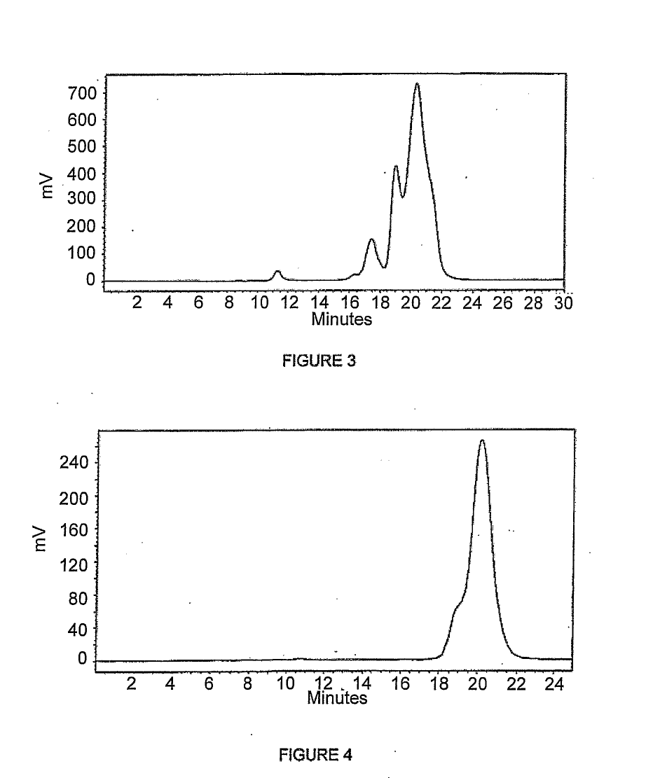

FIG. 3 provides the elution profile obtained from a 200 μL injection of a 300 mg/mL solution of sieved honey. Due to the honey being sieved before injection the glucose peak is reduced, compared to the fructose peak, instead of being in the roughly equal concentrations at which they are found in whole honeys (White et al., 1962).

Size exclusion matrices are commonly slightly hydrophobic and weakly anionic, which leads to non-ideal separation whereby separation is not strictly a function of molecular size (Cunico et at., 1998). This column uses styrene divinylbenzene as the size exclusion polymer, which may interact with phenolics allowing for ion exchange. Therefore, the retention time does not necessarily imply molecular size.

The eluant from the column was initially collected as four fractions:

| | | |
|---|---|---|
| 1 | 0.0-13.0 min | Phenolics |
| 2 | 13.0-18.4 min | Di- and oligosaccharides |
| 3 | 18.4-22.6 min | Glucose and Fructose |
| 4 | 22.6-30.0 min | Late eluting material |

The activity was consistently found in fractions between 18.4 and 30 min. Attempts were then made to confine all activity into one fraction.

TABLE 1
Activity of the fractions collected from the KS-2002 column

| | | Zone of | | | Phenol | | |
|---|---|---|---|---|---|---|---|
| Sample | | clearing a (mm) | | | equivalent b (%) | | |
| Fraction | Minutes | Average | SE | n | Average | SE | n |
| 1 | 0.0-13.5 | NA | — | 12 | NA | — | 2 |
| 2 | 13.0-18.4 | NA | — | 12 | NA | — | 2 |
| 3 | 18.4-22.6 | 11.19 | 0.09 | 12 | 10.71 | 0.03 | 2 |
| 4 | 22.6-30.0 | 15.07 | 0.16 | 12 | 17.06 | 0.12 | 2 |
| Whole | — | 18.72 | 0.18 | 20 | 24.67 | 0.92 | 2 |
| honey | | | | | | | |
| 1 | 0.0-13.5 | NA | — | 10 | NA | — | 2 |
| 2 | 13.5-18.4 | NA | — | 10 | NA | — | 2 |
| 3 | 18.4-21.5 | 15.47 | 0.27 | 10 | 17.63 | 0.40 | 2 |
| 4 | 21.5-25.0 | 17.79 | 0.13 | 7 | 21.91 | 0.54 | 2 |
| 5 | 25.0-30.0 | NA | — | 10 | NA | — | 2 |
| Sieved | — | 20.78 | 0.15 | 16 | 28.26 | 0.68 | 2 |
| Honey 1 | | | | | | | |
| 1-3 | 0.0-19.5 | NA | — | 10 | NA | — | 2 |
| 4 | 19.5-25.0 | 19.15 | 0.16 | 9 | 25.20 | 1.00 | 2 |
| Sieved | — | 19.15 | 0.12 | 16 | 26.86 | 0.25 | 2 |
| Honey 1 | | | | | | | |

Values are based on
a) individual wells over all plates, and
b) calculations of phenol activity on individual plates.

Table 1 shows the results of the trials of three different fraction collecting times, and the non-peroxide activity of the resulting fractions. Note that the first assay shown used whole honey, whereas the later two used sieved honey.

All the detectable activity was eventually confined to the fractions between 19.5 and 25.0 min. The 95% confidence intervals (CI,=mean ±2×SE) for these two fractions are given in Table 2.

The confidence intervals do not overlap for either the zones of clearing or phenol equivalents, which shows that the activity of fraction 4 is not statistically the same as that of the sieved honey. This indicates that some loss of activity has occurred, either by absorption onto the column or the partitioning into other fractions, at levels below the detection limits of the assay. The average amount of activity in fraction 4 was 90% of the sieved honey equivalent phenol activity.

TABLE 2
95% Cl for the zones of clearing and phenol equivalents
for the final trial of activity on the KS-2002 column

| Sample | Zone of clearing (mm) | Phenol equivalent (%) |
|---|---|---|
| Fraction 4 | 18.97-19.25 | 23.20-27.20 |
| Sieved Honey | 20.04-20.65 | 27.50-28.50 |

Values for zones of clearing were based on individual wells, and values for phenol equivalents was based on the calculated from plate data.

As can be seen from FIG. 3 this fraction contains almost all of the fructose. The monosaccharides themselves do not have antibacterial activity, except for the physical effect of reducing water activity and providing the substrate for glucose oxidase to produce H₂O₂ and gluconic acid. It is, therefore, evident that other compounds, in low concentrations, exist in this fraction.

A small peak at the tail of the fructose peak was observed by Snow (2001) in studies using an analytical version of this column. Attempts were made by Snow (2001) to isolate this fraction and identify its constituents through GC-MS and NMR. However, the large number of components in the sample meant that the results were inconclusive. Attempts were also made to correlate the size of this peak to the activity of the whole honey, although no relationship was found. This indicates that the peak is comprised of many compounds, where at least one is not active.

Snow (2001) noted that the peak correlated to this active fraction was UV active. This work could not identify these UV active peaks due to the preparative nature of the column reducing resolution, and interference from the high concentration of weakly UV active open chain monosaccharides in this region.

It was decided to re-inject fraction 4 to obtain a fraction free of monosaccharides. The spectrum of the re-injected fraction (FIG. 4) showed no evidence of a concentrated peak at the tail of the monosaccharide peak, although a new peak arising at 10.5 min was noted. The bulge at the beginning of the monosaccharide peak is likely to be residual glucose. An enlargement of the base line is shown in FIG. 5.

Sugar commonly increases the solubility of sparingly soluble molecules in aqueous systems. It is, therefore, feasible that there were compounds associated with, or solubilised by, the sugar. As the concentration of sugar declined, they disassociated and were eluted earlier.

It is possible that this peak could be the active factor, and the size of the peak is consistent with the idea that the compound (or compounds) responsible for the non-peroxide activity is found in exceptionally low concentrations. Alternatively it may be a degradation product, as the sample used had been stored for one month in the freezer.

A test of activity in the sample prior to being re-injected, however, showed no loss of activity, and this is supported by the findings that the active fraction is stable to being stored for in excess of 2 months in the freezer after isolation. It is possible that this peak may reflect degradation of a non antibacterial component.

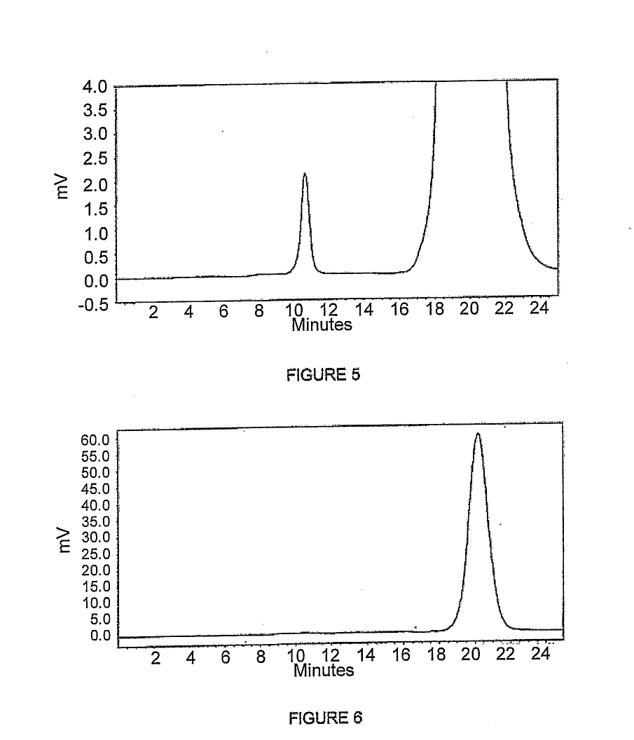

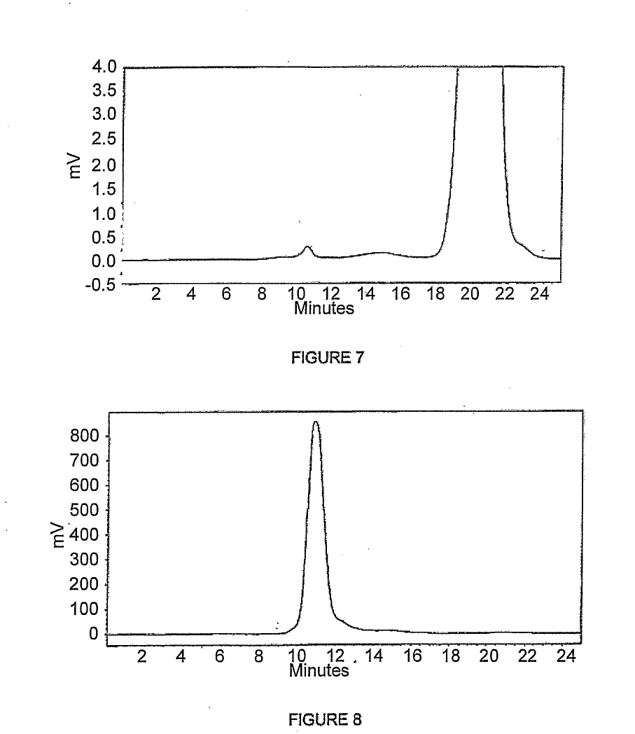

The 20.5 to 25.0 min fraction from the re-injection of fraction 4 was collected and re-injected to investigate whether the active peak observed by Snow (2001) could still be identified. It is evident that the new peak observed at 10.5 min also arises in the spectrum (FIG. 6 with an enlargement in FIG. 7), albeit much reduced.

This suggests that the peak was not a degradation product from storage and indeed may reflect the dissociation of the activity from the sugars.

A large enough quantity for a biological assay of the activity of this re-injected fraction (a minimum of 200 mg of honey passing through the column for a very rough indication of activity, or 800 mg for a rigorous assay of activity) was not collected.

It is not possible to say anything conclusive about the change in chromatographic behaviour following re-injection. It was decided not to pursue this avenue, but instead try a column with a different method of separation and greater capacity.

### EXAMPLE 4

### Semi-Preparative Fractionation of Honey (Reverse Phase)

The advantage of the C18 25 mm reversed phase column is that it has a substantially larger capacity whereby 1 g can be injected onto the column at a time, instead of the 60 mg that was injected onto the KS-2002 column.

The reversed phase preparative column used was three Delta-Pak C18 cartridges (25 mm×100 mm, 15 μm particle size, 100 Å pore size) in series. This was fitted with a Delta C18 guard insert (25 mm×10 mm, 15 μm particle size, 100 Å pore size). Chromatography was performed at room temperature at a flow rate of 10 mL/min with high loadings of 0.5 g/mL into a 2 mL loop.

Two different eluent systems were used. The first used only Milli-Q water. However, it was later decided to initially run with 100% Milli-Q water and then switch to 100% acetonitrile after the sugars were eluted. Due to the high flow rate used, detection was only possible using a wide-bore plumbed Waters 410 differential refractometer.

Fractions collected in water were freeze-dried In large evaporating dishes. Fractions collected in MeCN were concentrated under reduced vacuum and then drying was completed on the freeze drier.

This greatly reduced the amount of time spent collecting sufficient sample for the biological assay of activity. Additionally, it was of interest as to whether this column could more effectively resolve the activity from the monosaccharides. FIG. 8 shows the spectrum obtained from using this column running in Milli-Q water at high column loadings (0.5 g/mL, 2 mL injection).

This spectrum is dominated by the monosaccharides peak. The column was not able to resolve glucose from fructose, but it does show a set of peaks eluting just after the monosaccharides, around 14 to 25 min.

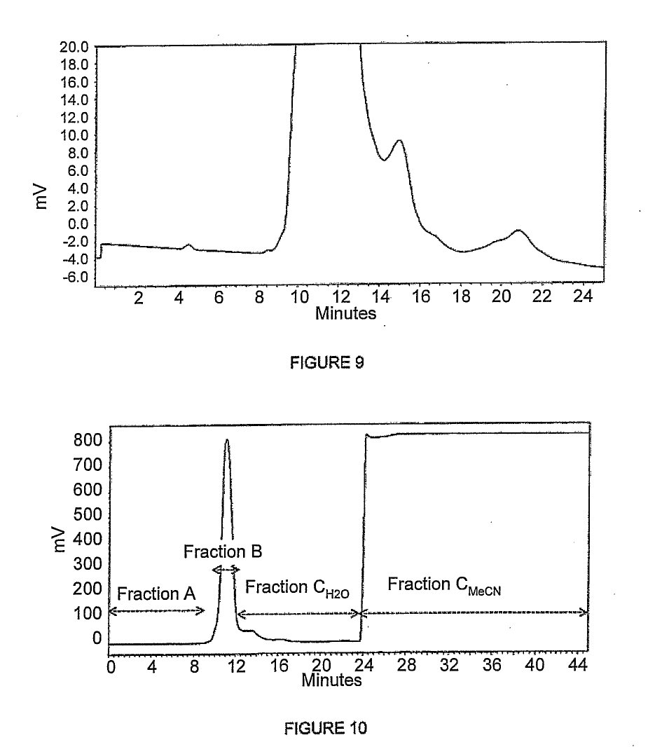

Some of these peaks can be attributed to oligosaccharides, which are present in active manuka honey at levels of around 8% of the total honey (Weston and Brocklebank, 1999). FIG. 9 shows an enlargement of the baseline demonstrating these peaks.

The eluant from the column was initially collected as four fractions:
A 0.0 to 8.3 min Early eluting material
B 8.3 to 11.8 min Sugars
C 11.8 to 25.0 min Later eluting material

It was found that all detectable activity eluted in fraction C (Table 3).

TABLE 3
Activity of fractions initially collected from the C18 column.

| | | Zone of | | | Phenol | | |
|---|---|---|---|---|---|---|---|
| | Fraction | clearing ^{a} (mm) | | | equivalent ^{b} (%) | | |
| Sample | (min) | Average | SD | n | Average | SD | n |
| Fraction A | 0.0-8.2 | NA | — | 16 | NA | — | 2 |
| Fraction B | 8.2-11.8 | NA | — | 17 | NA | — | 2 |
| Fraction C | 11.8-30.0 | 17.51 | 1.31 | 8 | 21.10 | 2.40 | 2 |
| Sieved | — | 20.91 | 0.48 | 15 | 28.15 | 0.21 | 2 |
| Honey 5 | | | | | | | |

Values are based on
^{a} individual wells over all plates, and
^{b} calculations of phenol activity on individual plates.

It was of interest whether some of the activity could be eluted by MeCN as this would show differential solubility of the fraction. Additionally, it was necessary to determine whether all the activity was being eluted from the column with Milli-Q water.

MeCN is a stronger solvent than H₂O in reversed phase chromatography and so it can be used to flush any residual material from the column. The fractions were collected as indicated in FIG. 10.

The column was initially run with Milli-Q water and fractions A and B were collected as previously outlined. At the cross-over from fraction B to fraction C (11.8 min), the pump was stopped, and the solvent was swapped directly over to 100% MeCN.

Fraction C_{H2O} was collected from 11.8 min until the MeCN reached the detector (approximately 13 min or at the retention time of 24 min), as indicated by a rapid rise in the base line caused by the change in refractive index of the solvent. From this time, three column volumes of MeCN were flushed through the column, and the eluent collected as fraction C_{MeCN}.

As Table 4 shows, all the detectable activity was retained in fraction C_{H2O} and none was associated with C_{MeCN}. This shows that the activity is not being reversibly retained by the column.

Fraction C and C_{H2O} only accounted for 72% and 75% of the sieved honey equivalent phenol activity respectively, with no other fractions displaying detectable activity. This suggests that the activity could have become irreversibly bound on the column.

The column used was preparative and consisted of C18 chains on a non-endcapped silica support and it is possible that material could have adhered to the silica, or even been decomposed by reaction with active silanol groups. Additionally, the fraction consistently gave varying levels of partial activity. In this particular case it could have arisen from the lack of diffusibility of the non-peroxide activity in the assay when the sugar content was reduced.

TABLE 4
Weights and activity of fractions from reversed phase chromatography on the C18 column.

| | Fraction | Weight ^{a} (% of dry weight) | | | Zone of clearing ^{b} (mm) | | | Phenol equivalent ^{c} (%) | | |
|---|---|---|---|---|---|---|---|---|---|---|
| Sample | (min) | Average | SE | n | Average | SD | n | Average | SD | n |
| Fraction A | 0.0-8.2 | negligible | — | 4 | NA | — | 36 | NA | | 4 |
| Fraction B | 8.2-11.8 | 86.27 | 1.25 | 4 | NA | — | 36 | NA | — | 4 |
| Fraction | 11.8-27.0 | 9.52 | 2.91 | 4 | 17.75 | 0.33 | 28 | 19.98 | 1.45 | 4 |
| C(H₂O) | | | | | | | | | | |
| Fraction | 27.0-45.0 | 2.14 | 0.04 | 2 | NA | — | 36 | NA | — | 4 |
| C(MeCN) | | | | | | | | | | |
| Sieved | — | — | — | — | 20.83 | 0.09 | 24 | 27.91 | .2.40 | 3 |
| Honey 5 | | | | | | | | | | |
| Sieved | — | — | | | 20.16 | 0.13 | 10 | 23.91 | 0.27 | 2 |
| Honey 6 | | | | | | | | | | |

Values are based on
^{a} replicated HPLC experiments,
^{b} individual wells over all plates, and
^{c} calculations of phenol activity on individual plates.

### Comparison of the KS-2002 and C18 Columns

The Delta-Pak C18 active fraction incorporated far less sugar that the KS-2002 active fraction, and reflects a better separation of the activity from the sugars. Conversely, a greater proportion of the activity was recovered from the KS-2002 column, which may reflect chemical interactions of the active material with the silica support in the C18 column.

Full activity was observed in the active fraction from the KS-2002 column as opposed to the partial activity from the C18 column fraction. However, this may be related to the greater sugar content of the KS-2002 fraction aiding diffusion on the assay.

### EXAMPLE 5

### Stability of Non-Peroxide Activity in UMF Containing Fractions

Previous authors have noted that honeys with non-peroxide activity have a significant activity remaining after the honey is heated (Bogdanov, 1984; Molan and Russell, 1988; Roth et al., 1986) or stored (Bogdanov, 1984; Sealey, 1988), and this was part of the observation which lead to the proposal of non-peroxide activity.

This study looked at the stability in the isolated HPLC fraction while being stored for short periods of time (8 hrs to 24 hrs) at room and refrigeration temperature, and for moderate time periods (1 to 8 weeks) at freezer temperature.

This was of practical concern as in the course of experiments, we want to be certain that the fraction is not degrading and therefore that any loss of activity is due to removal of non active components. Additionally, it was useful to know how much sample could be “stock piled” for use in testing.

A side interest was to see whether activity could, in fact, increase in storage due to the anecdotal evidence in industry suggesting that storage can increase the activity of the whole honey.

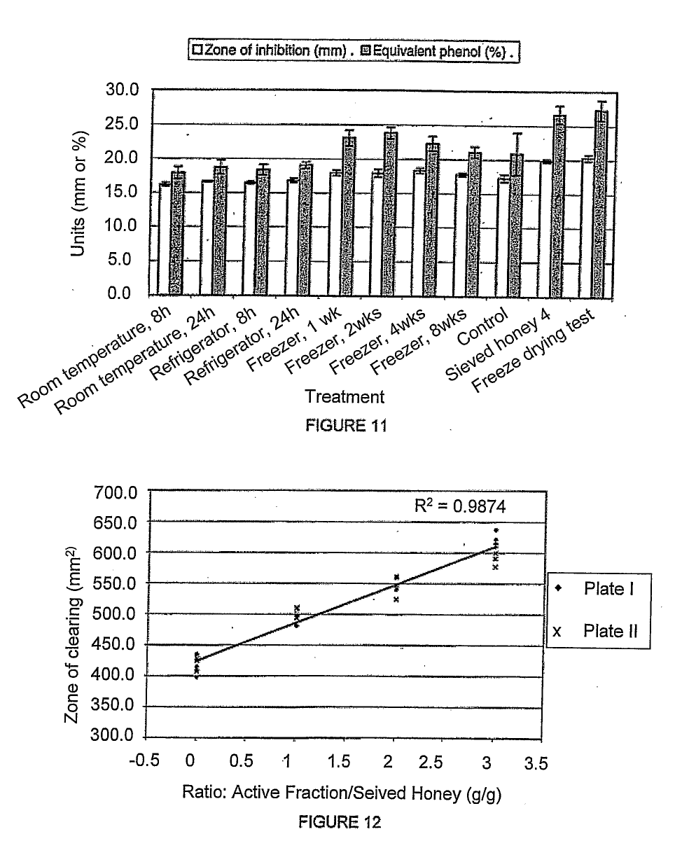

The summary results for this experiment are given Table 5 and the results are shown visually in FIG. 11.

TABLE 5
Summary data from the stability trial.

| | Zones of clearing ^{a} (mm) | | | Phenol equivalent ^{b} (%) | | |
|---|---|---|---|---|---|---|
| Treatment | Average | SE | n | Average | SE | n |
| Room | 16.18 | 0.14 | 8 | 17.88 | 0.45 | 2 |
| temperature, | | | | | | |
| 8 h | | | | | | |
| Room | 16.65 | 0.05 | 8 | 18.75 | 0.52 | 2 |
| temperature, | | | | | | |
| 24 h | | | | | | |
| Refrigerator, | 16.44 | 0.09 | 8 | 18.38 | 0.38 | 2 |
| 8 h | | | | | | |
| Refrigerator, | 16.82 | 0.15 | 8 | 19.06 | 0.24 | 2 |
| 24 h | | | | | | |
| Freezer, | 17.95 | 0.20 | 8 | 23.06 | 0.57 | 2 |
| l wk | | | | | | |
| Freezer, | 17.92 | 0.29 | 8 | 23.83 | 0.42 | 2 |
| 2 wks | | | | | | |
| Freezer, | 18.33 | 0.19 | 7 | 22.29 | 0.52 | 2 |
| 4 wks | | | | | | |
| Freezer, | 17.78 | 0.14 | 8 | 21.06 | 0.41 | 2 |
| 8 wks | | | | | | |
| Control | 17.28 | 0.27 | 34 | 20.86 | 1.55 | 8 |
| Sieved | 19.88 | 0.11 | 72 | 26.62 | 0.65 | 10 |
| Honey 4 | | | | | | |
| Freeze | 20.34 | 0.23 | 17 | 27.27 | 0.72 | 4 |
| drying test | | | | | | |

Values are based on
^{a} individual wells over all plates, and
^{b} calculations of phenol activity on individual plates.

Sieved honey was separated on the KS-2002 column and the fraction between 19.5 to 25 min was collected and transferred immediately to the freezer. At the completion of injections for the day the fractions were freeze-dried.

Once dried they were reconstituted to 50% strength with distilled water, and then stored for a range of times at either room temperature, or in the refrigerator or freezer. Each sample was equivalent to 480 mg of sieved honey.

After storage, samples were diluted to 25% strength and tested in four wells on duplicated plates to investigate whether any toss or gain of activity occurred. These results were compared to the activity of the freshly collected active fraction tested on the same plate. Additionally two replicates of sieved honey were freeze-dried to investigate the effect of the freeze-drying process on the activity of the honey. The percentage retention of original fraction is given in Table 6. The retention of the original activity ranged from 94 to 106% in the zones of the clearing, and from 86 to 114% in the phenol equivalents. Therefore, the various forms of storage have had some effect on non-peroxide activity in this isolated active fraction.

TABLE 6
Retention of non-peroxide activity in stored fractions.
Percentage of the original activity.

| | Zone of clearing ^{a} | Phenol equivalent ^{a} |
|---|---|---|
| Storage conditions | (%) | (%) |
| Room temperature, 8 h | 93.7 | 85.7 |
| Room temperature, 24 h | 96.3 | 89.9 |
| Refrigerator, 8 h | 95.2 | 88.1 |
| Refrigerator, 24 h | 97.3 | 91.4 |
| Freezer, 1 wk | 103.9 | 110.5 |
| Freezer, 2 wks | 103.8 | 114.2 |
| Freezer, 4 wks | 106.1 | 106.9 |
| Freezer, 8 wks | 102.9 | 100.9 |

Table 7 provides the REML analysis of these results. These results show that some of these changes in activity on storage are statistically significant. Using the zones of clearing data, the room temperature and refrigerator samples are all significantly lower than the control.

The phenol equivalent does not show statistical significance in these treatments, probably due to there being less replicates in this data set and so the larger standard error obscures any difference between the predicted means.

A statistically significance increase in activity was also seen in the zones of clearing data for the eight week freezer fraction, and in the phenol equivalent data for the four week freezer fraction.

A limitation in this experiment, however, was that treatments were not conducted in replicate, and were tested in duplicate plates on the same day. Consequently, day-to-day variation may play a large role in determining significance. The use of the phenol standard curve to generate the phenol equivalents appears to give an even wider range of values than using zones of clearing, suggesting that phenol standards are not compensating for these day-to-day effects.

Despite the statistical significance of these results, a good retention of activity is still observed. There is also statistically significant evidence of increasing activity in some samples stored in the freezer. However, the influence of day-to-day and plate-to-plate effects in this experiment remains unknown due to the treatments not being replicated.

TABLE 7
Statistical information about the significance of the changes in
non-peroxide activity following storage of the active HPLC fraction.

| | Zone of clearing ^{a} (mm) | | | Phenol equivalent ^{b} (%) | | |
|---|---|---|---|---|---|---|
| | Difference | | | Difference | | |
| | between | | Signif- | between | | Signif- |
| Treatment | the means | 2 × SE | icant? | the means | 2 × SE | icant? |
| Room | −1.87 | 0.78 | Yes | −1.46 | 7.68 | No |
| temper- | | | | | | |
| ature, | | | | | | |
| 24 h | | | | | | |
| Room | −2.33 | 0.78 | Yes | −2.33 | 7.68 | No |
| temper- | | | | | | |
| ature, | | | | | | |
| 8 h | | | | | | |
| Refrig- | −2.07 | 0.78 | Yes | −1.83 | 7.68 | No |
| erator, | | | | | | |
| 8 h | | | | | | |
| Refrig- | −1.7 | 0.78 | Yes | −1.15 | 7.68 | No |
| erator, | | | | | | |
| 24 h | | | | | | |
| Freezer, | 0.28 | 0.46 | No | 1.96 | 2.88 | No |
| l wk | | | | | | |
| Freezer, | −0.49 | 0.62 | No | −0.5 | 3.44 | No |
| 2 wks | | | | | | |
| Freezer, | 0.44 | 2.5 | No | 3.71 | 2.84 | Yes |
| 4 wks | | | | | | |
| Freezer, | 0.85 | 0.6 | Yes | 3.18 | 3.44 | No |
| 8 wks | | | | | | |

Difference between the means is given by: predicted mean of the treatment - predicted mean of the control. Values are based on
^{a} individual wells over all plates, and
^{b} based on calculations of phenol activity on individual plates.

### EXAMPLE 6

### Fortification of Honey

Honeys with exceptionally high antibacterial activity do not routinely occur naturally, and as a result, they command high prices. Therefore, a commercial advantage exists if a method could be developed to concentrate the activity of honeys that are too low in activity to be medically useful, or to increase the potency of an already highly active honey to a level that cannot be routinely obtained naturally.

This is only useful for concentrating non-peroxide activity (UMF) as peroxide activity of honey is produced in situ and depends on the concentrations of peroxide destroyers and stability of the glucose oxidase.

As reversed phase HPLC was able to resolve the UMF and the column could cope with large loadings of sample, fraction C (eluted with 100% Milli-Q water), as opposed to fraction 4 on the size exclusion/ion exchange, was collected and used to fortify sieved honey.

To generate the fortified samples, fraction C (produced from the separation of 1 g of sieved honey), was added to 1 g of unprocessed sieved honey in 1 mL of distilled water. This 50% solution was further diluted with catalase to give a 25% solution. Therefore, samples technically have twice the amount of UMF as the sieved honey control.

An initial experiment (Table 8) showed that a small increase over and above the activity of the sieved honey was achieved.

TABLE 8
Initial investigation of the fortification of sieved honey

| | Zone of clearing ^{a} (mm) | | | Phenol ^{b} (%) | | |
|---|---|---|---|---|---|---|
| Sample | Average | SE | n | Average | SE | n |
| Sieved honey/A | 22.68 | 0.14 | 16 | 28.75 | 1.26 | 2 |
| Fraction C (1:1) B | 22.52 | 0.10 | 15 | 28.43 | 1.01 | 2 |
| Sieved Honey 6 | 20.16 | 0.13 | 10 | 23.91 | 0.27 | 2 |
| Fraction C | 19.12 | 0.33 | 10 | 21.19 | 0.74. | 2 |

Values are based on
^{a} individual wells over all plates, and
^{b} calculations of phenol activity on individual plates

This accounted for a 23.5% increase in equivalent phenol activity of the sieved honey. Since a doubling of the activity was not observed, this experiment was expanded to include increasing levels of fortification to investigate if a directly proportional response to increasing dose of the active fraction was possible.

Each dose consisted of the addition of fraction C at a concentration equivalent to that found in 1, 2 and 3 g of sieved honey, to 1 g of unprocessed sieved honey as described above.

Table 9 shows the activity of the resulting samples and the zone of clearing results are shown graphically in FIG. 12.

TABLE 9
Investigation of the proportionality of fortifying sieved honey

| | Zone of clearing ^{a} (mm) | | | Phenol ^{b} (%) | | |
|---|---|---|---|---|---|---|
| Sample | Average | SE | n | Average | SE | n |
| 1:3 | 24.60 | 0.14 | 8 | 33.61 | 0.87 | 2 |
| Sieved honey/1:2 | 23.44 | 0.10 | 8 | 31.00 | 0.40 | 2 |
| Fraction C 1:1 | 22.33 | 0.07 | 8 | 28.64 | 0.00 | 2 |
| Sieved Honey 7 | 14.87 | 0.11 | 8 | 15.75 | 0.34 | 2 |
| Fraction C | 20.39 | 0.11 | 8 | 24.80 | 0.05 | 2 |

Values are based on
^{a} individual wells over all plates, and
^{b} calculations of phenol activity on individual plates

A strong correlation (R2=0.9874) is seen indicating that a directly proportional response is occurring. This identifies that it is possible to increase the non-peroxide activity of honey.

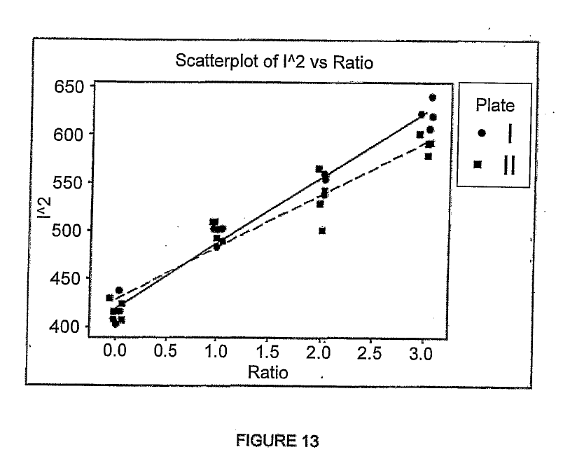

Linear regression analysis, however, showed that the duplicate plates used in the testing of these samples had statistically significant differences in gradient (p value=0.011). These curves can be seen in the Minitab output in FIG. 13.

It is not unusual to see plate effects such as these. As already discussed, plate variation is one of the most significant variables in the WDA method and consequently all samples were tested on duplicate plates, which were averaged to compensate for this effect.

Alternatively, the data points at 1:3 fortification (ratio 3, FIG. 13) show more variation between the two curves compared to the other three fortification levels. It is, therefore, possible that some factor, limited to this point may be skewing the curve.

This could simply be due to this sample not being homogenous when added to the two plates. However, the assay is not as sensitive at high activities. The reasons for this are three fold:
(i) The zones of clearing can overlap which tends to cause the zones of clearing to elongate, making measurement difficult.
(ii) Diffusion is more variable.
(iii) The zone of clearing can exceed the highest standard.

In this study the fractions were carefully spaced to minimise the chance of the zones of clearing overlapping, however, the 1:3 fortification sample was approaching the activity of the highest standard.

Irrespective of these considerations a method for the preparation of a UMF containing fraction of manuka honey and its use in the fortification of honey has been demonstrated.

Where in the foregoing description reference has been made to integers or components having known equivalents then such equivalents are herein incorporated as if individually set forth.

Although the invention has been described by way of example and with reference to possible embodiments thereof it is to be appreciated that improvements and/or modification may be made thereto without departing from the scope or spirit of the invention.

### REFERENCES

Abuharfeil, N.; Al-Oran, R.; Abo-Shehada, M. (1999) The effect of bee honey on proliferative activity of human B- & T-lymphocytes and the activity of phagocytes Food & Agri. Invn. 11, 169-177
Adcock, D. (1962) The effect of catalase on the inhibine and peroxide values of various honeys J. Apic. Res. 1, 38-40
Allen, K. L.; Molan, P. C.; Reid, G. M. (1991) A Survey of the Antibacterial Activity of Some New Zealand Honeys J. Pharm. Pharmacol. 43, 817-822
Benj B.; Sunshine, G.; Leskowitz, S. (1996) Immunology 3rd edition New York Wiley-Liss
Bogdanov, S. (1997) Nature and Origin of the Antibacterial Substances in Honey Lebensm-Wiss. U.-Technol. 30, 1, 748-753
Breitmaier, B.; Voelter, W. (1987) Carbon-13 NMR Spectroscopy 3rd edition Verlagsgesellschaft, Weinheim, Germany
Bulman, M. W. (1955) Honey as a surgical dressing Middlesex Hospital Journal 5, 188-189
Chirife, J.; Scarmato, G.; Herszagel, L. (1982) Scientific basis for use of granulated sugar in treatment of infected wounds Lancet, March 6, 560-561
Cizmarik, L; Matel, I. (1970) Examination of the chemical composition of propolis. I. Isolation and identification of the 3 4-dihydroxycinnamic acid (caffeic acid) from propolis Experientia 26, 7, 713
Cizmarik, L; Matel, I. (1973) Chemical composition of propolis. 2. Isolation and identification of 4-hydroxy-3-methoxycinnamic acid (ferulic acid) from propolis J. Apicult. Res. 12, 1, 52-54
Cooper, R. A.; Molan, P. C.; Harding, K. G. (1999) Antibacterial activity of honey against strains of Staphylococcus aureus from infected wounds Journal of the Royal Society of Medicine 92, 283-285
Cotton, F A, Wilkinson, G F R S (1972) Advanced inorganic chemistry A comprehensive text 3rd edition, John Wiley & Sons, Inc., USA
Dustmann, J H (1971) Catalase activity in honey of heather plant (Ericacea) flow Z Lebensm-Unters-Forsch 145, 5, 294-5
Dustmann, J H (1979) Antibacterial effect of honey Apiacta 14 7-11
Efem, S B B (1988) Clinical Observations on the Wound Healing Properties of Honey Brit J Surgery 75, 679-681
Efem, S B E (1993) Recent Advances in the Management of Fournier's Gangrene Preliminary Observations Surgery 113, 2, 200-204.
El-Banby, M, Kandil, A, Abou-Sehly, G, El-Shenf, M E, Abel-Wahed, K (1989) Healing effect of floral honey and honey from sugar-fed bees on surgical wound (animal model) Fourth International Conference on Apiculture in Tropical Climates, Cairo, International Bee Research Association London, 46-49
Farr, J M. (2005*) An Investigation into some properties of the non-peroxide antibacterial activity of manuka honey Waikato University Masters Thesis (* embargoed).
Ferreres, F, Tomas-Barberan, F A, Soler, C, Garcia-Viguera, C, Ortiz, A, Tomas-Lorentz, F (1994) A simple extractive technique for honey flavonoid BIPLC analysis Apidologie 25, 1, 21-30
Ghisalbert, E L (1979) Propolis A review Bee World 60, 59-84
Gonnet, M.; Lavie, P. (1960) The influence of heat on the antibiotic factor present in Honey Ann. Abeille 34, 349-364
Gupta, S. K.; Singh, H.; Varshney, A. C.; Prakash, P. (1992) Therapeutic efficacy of honey in infected wounds in buffaloes Indian Journal of Animal Science 62, 6, 521-523
Hodges, R.; White, E. P. (1966) Detection and isolation of tutin and hyenanchin in toxic honey New Zealand J. Sci. 9, 1, 233-35
Hutton, D. J. (1966) Treatment of pressure sores Nursing Times 62, 46, 1533-1534
Itoh, K.; Amamiya, I.; Ikeda, S.; Konishi, M. (1994) Anti-Helicobacter pylori substances in propolis Honeybee Science 15, 4, 171-173
Jones, K. P.; Blair, S.; Tonks, A.; Price, A.; Cooper, R. (2000) Honey and the stimulation of inflammatory cytokine release from a monocytic cell line First World Wound Healing Congress, Melbourne, Australia
Kandil, A.; El-Bnby, M.; Abdel-Wahed, K.; Abou-Sehly, G.; Ezzat, N. (1987) Healing effect of true floral and false non-floral honey on medical wounds J. Drug Res. (Cairo) 17, 1-2, 71-75
Lavie, P. (1960) Antibacterial substances in the honeybee colony Ann. Abaille 3, 103-183
Majno, G. (1975) The Healing Hand. Man and Wound in the Ancient World Cambridge, Mass., Harvard University Press
Marcucci, M. C. (1995) Propolis: Chemical Composition, Biological Properties and Therapeutic Activity Apidologie 26, 89-99
Marhuenda Requena E.; Alarcon de la Lastra, C.; Garcia Gimenez, M. D. (1987) Demonstration of antibacterial properties of phenolic acids from Thymus carnosus Boiss. Isolation of caffeic, vanillic, p-coumaric, p-hydroxybenzoic and syringic acids Plant. Med. Phytother. 21, 2, 153-9
Maurizio, A. (1962) From the raw material to the finished product: Honey Bee World 43 66-81
McInemey, R. J. F. (1990) Honey—a remedy rediscovered Journal of the Royal Society of Medicine 83, 127
Mladenov, S. (1974) Present problems of Apitherapy. Hive products: Food health and beauty Proceedings of the international Symposium on Apitherapy Apimonida Publishing House, Madrid
Molan, P. C.; Russell, K. M.; (1988) Non-peroxide antibacterial activity in some New Zealand Honeys J. Apic. Res. 27, 1, 62-67
Molan, P. C. (1992a) The antibacterial activity of honey 1. The nature of the antibacterial activity Bee World 73, 1, 5-28
Molan, P. C. (1992b) The antibacterial activity of honey 2. Variation in the potency of the antibacterial activity Bee World 73, 2, 59-76
Molan, P. C. (2001) Why honey is effective as a medicine 2. The scientific explanation of its effects Bee World 82, 1, 22-40
Morales, C. F. (1991) Margarodidae (Insecta:Hemiptera Fauna of New Zealand/Ko te Aitanga Pepeke o Aotearoa 21, 16-17
Oryan, A.; Zaker, S. R.; (1998) Effects of topical application of honey on cutaneous wound healing in rabbits J. Vet. Med. Series A 45, 3, 181-188
Perry, N. B.; Brennan, N. J.; Van Klink, J. W.; Harris, W.; Douglas, M. H.; McGimpsey, J. A.; Smalifield, B. M.; Anderson, R. E. (1997) Essential oils from New Zealand manuka and kanuka: chemotaxonomy of Leptospermum Phytochemistry 44, 8, 1485-1494
Ransome, H. M. (1937) The sacred bee in ancient times and folklore London, George Allen and Unwin
Rivera-Vargas, L. I.; Schmitthenner, G. F.; Graham, T. I. (1993) Soybean flavonoids effects on and metabolism by Phytophthora sojae Phytochemistry 32, 4, 851-857
Russell, K. M. (1983) The antibacterial properties of honey Waikato University Masters Thesis
Russell, K. M.; Molan, P. C.; Wilkins, A. L.; Holland, P. T. (1990) Identification of Some Antibacterial Constituents of New Zealand Manuka Honey J. Agric. Food Chem. 38, 1, 10-13
Schepartz, A. I. (1966) Honey catalase: occurrence and some kinetic properties J. Apic. Res. 5, 3, 167-176
Schepartz, A. I.; Subers, M. H. (1966) Catalase in honey J. Apic. Res. 5, 1, 37-43
Schuler, R.; Vogel, R. (1956) Active ingredients of bee honey. I Arzneimittel-Forsch. 6, 661-663
Snow, M J. (2001*) An Investigation into the non-peroxide antibacterial activity of manuka honey Waikato University Masters Thesis (* embargoed).
Steeg, E.; Montag, A. (1988) Minor components of honey with significance for the aroma. II. Organoleptic decomposition products of aromatic carboxylic acids and glycosidically bound aromatic compounds Dtsch. Lebensm.-Rundsch. 84, 5, 147-150
Stinson, E. E.; Subers, M. H.; Petty, J.; White J. W., Jr. (1960) The composition of honey V. Separation and identification of the organic acids Archives of Biochemistry and Biophysics 89, 6-12
Tan, S. T.; Holland, P. T.; Wilkins, A. L.; Molan, P. C. (1988) Extractives from New Zealand Honeys. 1. White Clover, Manuka, and Kanuka Unifloral Honeys J. Agric. Food Chem. 36, 453-460
Tan, S. T.; Wilkins, A. L.; Molan, P. C.; Holland, P. T.; Reid, M. (1989) A Chemical Approach to the Determination of Floral Sources of New Zealand Honeys J. Apic. Res. 28, 4, 212-222
Thimann, K. V. (1963) The life of bacteria 2^{nd} edition New York, Macmillan
Verge, J. (1951) The antibacterial activity of propolis in honey and royal jelly Apiculteur 95, section scientifique, 13-20
Wahdan, H. A. L. (1998) Causes of the Antimicrobial Activity of Honey Infection 26, 1, 26-31
Ward, C. (2000) Kanuka and Manuka Forest & Bird 295 24-27
Weston, R. J.; Brocklebank, L. K. (1999) The oligosaccharide composition of some New Zealand honeys Food Chemistry 64, 33-37
Weston, R. J.; Brocklebank, L. K.; Lu, Y. (2000) Identification and quantitative levels of antibacterial components of some New Zealand honeys Food Chemistry 70, 427-435
Weston, R. J.; Mitchell, K. R.; Allen, K. L. (1999) Antibacterial phenolic components of New Zealand manuka honey Food Chemistry 64, 295-301
Weston, R. J. (2000) The contribution of catalase and other natural products to the antibacterial activity of honey: A review Food Chemistry 71, 235-239
White, J. W. Jr.; Riethof, M. L.; Suber M. H.; Kushnir, I. (1962) Composition of American Honeys Technical Bulletin No 1261 U S Government Printing Office, Washington D.C.
White, L W., Jr.; Subers, MR.; Schepartz, A. J. (1963) The identification of inhibine, the antibacterial factor in honey as hydrogen peroxide and its origin in a honey glucose oxidase system Biochem. Biophys. Acta 73, 57-70
White, L W. Jr. (1975) “of Honey” Honey: A comprehensive survey E Crane, Ed London, Heinemann, 157-206
White, J W, Jr (1978) Honey [in food research] Adv Food Res 24, 287-374
White, J W Jr (1992) The hive and the honey bee Ch 21 869-925
Wilkins, A L, Lu, Y, Molan, P C (1993) Extractable organic substances from New Zealand unifloral manuka (Leptospermum scoparium) honeys J Apic Res 32, 1,
Wu, J. (2000) A chemical investigation of oligosaccharides in some New Zealand unifloral honeys Waikato University Masters Thesis
Websites: www Airborne Honey Ltd, P0 Box 28, Leeston, New Zealand

1-30. (canceled)

31. A Unique Manuka Factor (UMF) fortified honey.

32. The UMF fortified honey of claim 31 where the UMF value of the honey is greater than 25.

33. The UMF fortified honey of claim 32 where the UMF value of the honey is greater than 35.

34. The UMF fortified honey of claim 33 where the honey is manuka honey.

35. A method of preparing a UMF fortified honey including the step of:
a) mixing a honey with a UMF containing fraction; where the UMF containing fraction is substantially free of monosaccharide sugars.

36. The method of claim 35 where the honey is manuka honey.

37. A method of preparing a UMF containing fraction including the steps of:
a) applying an amount of manuka honey to a C18 chromatography matrix in the format of a column;
b) eluting the sample from the matrix with water; and
c) collecting a UMF containing fraction; where the UMF containing fraction is substantially free of monosaccharide sugars.

38. The method of claim 37 where the matrix has a 15 μm particle size and a 100 Å pore size.

39. A UMF containing fraction of manuka honey where the fraction is substantially free of monosaccharide sugars.

40. The UMF containing fraction of claim 39 having antibacterial activity, where the antibacterial activity of the UMF containing fraction is labile at a pH greater than 39.

41. The UMF containing fraction of claim 40 where the antibacterial activity has a retention time of 19.4 to 25 minutes when a sample (20 μL) of honey containing the UMF containing fraction is applied to Shodex™ Sugar KS-801 and KS-802 analytical columns in series and in the sodium form, operated at a temperature of 50° C. and eluted with Milli-Q water at a rate of 1 mL/min.

42. The UMF containing fraction of claim 42 where the antibacterial activity has a retention time of 19.4 to 21.7 minutes.

43. A medicament comprising the UMF containing fraction of claim 39 and a pharmaceutically-acceptable carrier.

44. The medicament of claim 43 where the medicament is a wound dressing.

45. A food stuff comprising the UMF containing fraction of claim 39 and an edible product.
